(19) United States
(12) Patent Application Publication
(10) Pub. No.: US 2020/0107817 A1
(43) Pub. Date: Apr. 9, 2020

(54) METHOD FOR IMAGING AN AREA OF A MEDIUM WITH ULTRASOUND CONTRAST AGENTS AND ASSOCIATED DEVICE

(71) Applicants: INSTITUT NATIONAL DE LA SANTE ET DE LA RECHERCHE MEDICAL (INSERM), Paris (FR); ECOLE SUPERIEURE DE PHYSIQUE ET DE CHIMIE INDUSTRIELLES DE LA VILLE DE PARIS, Paris (FR); UNIVERSITÉ PARIS DIDEROT - PARIS 7, Paris (FR); CENTRE NATIONAL DE LA RECHERCHE SCIENTIFIQUE, Paris (FR); SORBONNE UNIVERSITÉ, Paris (FR)

(72) Inventors: Jean PROVOST, PARIS (FR); Mickael TANTER, PARIS (FR); Béatrice BERTHON, PARIS (FR)

(21) Appl. No.: 16/495,562

(22) PCT Filed: Mar. 22, 2018
(86) PCT No.: PCT/EP2018/057253

§ 371 Date: Sep. 19, 2019

(30) Foreign Application Priority Data
Mar. 22, 2017 (EP) 17305321.6

(51) Int. Cl. A61B 8/08; A61B 8/00

(52) U.S. Cl. CPC A61B 8/481; A61B 8/0808; A61B 8/14; A61B 8/08; A61B 8/54; A61B 8/0883

(57) ABSTRACT

The present invention relates to the field of acoustoelectric and acoustooptical imaging methods. It is known a specific example of an acoustoelectric imaging method, in which focused ultrasonic waves are emitted so as to form an image of the current, line by line. However, the acquisition process disclosed is slow, and all the more so because, as the resulting electrical signals are very weak, a high level of averaging is required. Low frame rates are therefore obtained. That is why the inventors worked on an imaging method with improved contrast and resolution. The present invention proposes a method for imaging a medium (10) wherein ultrasound contrast agents (12) are present.

### TECHNICAL FIELD OF THE INVENTION

The present invention concerns a method for imaging a medium with ultrasound contrast agents and an associated device.

### BACKGROUND OF THE INVENTION

Electrical impulses travel through organs of a subject, such as the heart or muscles. Such electrical impulses convey various information, notably orders relative to the operating of the organ. For instance, an electrical impulse may trigger the contraction of a muscle. These orders stem from the brain of the subject.

From this, it results that a better knowledge of electrical impulses would provide a better understanding of brain mechanisms as well as another way of assessing the proper operating of an organ, and hence the diagnostic of illness. As a specific example, imaging the electrical activation of biological tissues is crucial in applications such as cardiology, to provide better understanding for the diagnosis and treatment of pathologies such as arrhythmias. It is therefore desired to image the propagation of the electrical impulses.

For this, it is known to achieve acoustoelectric imaging which has emerged as a promising technique for direct imaging of current densities in-vivo. Acoustoelectric imaging exploits the interaction between ultrasound and electric currents to determine the value of the electric current at points of interaction between ultrasound and tissue, typically at the focal spot of a focused ultrasonic wave. Document U.S. Pat. No. 8,057,390 discloses a specific example of an acoustoelectric imaging method, in which focused ultrasonic waves are emitted so as to form an image of the current, line by line.

However, the acquisition process disclosed in document U.S. Pat. No. 8,057,390 is slow, and all the more so because, as the resulting electrical signals are very weak, a high level of averaging is required. Low frame rates are therefore obtained.

### SUMMARY OF THE INVENTION

There is thus a need to provide an imaging technique which enables to map a physical quantity of a medium, notably current densities, with improved resolution and resolution.

The specification proposes a method for imaging a medium wherein ultrasound contrast agents are present, the method comprising the step of applying at least one ultrasound wave to the medium, each ultrasound wave propagating up to the region of interest, to collect each wave transmission of this at least one ultrasound wave. The method also comprises a step of measuring a physical quantity of the medium, the physical quantity being affected by the or each applied ultrasound wave in the medium, the physical quantity being an electric quantity or an optical quantity, and a step of imaging the physical quantity in the medium based on each wave transmission and the measured physical quantity.

In other words, the specification proposes an acousto-optic imaging method or an acoustoelectric imaging method of a medium wherein ultrasound contrast agents are present.

In such method for imaging, the ultrasound contrast agents are used as a contrast enhancement. This results in an increased sensitivity of the method for imaging. As a consequence, such method for imaging is an imaging technique which enables to map a physical quantity of the medium, notably current densities, with improved resolution and contrast.

In other words, the step of imaging enables to obtain an image or a map with a quality enhanced by the presence of ultrasound contrast agents.

According to further aspects of which are advantageous but not compulsory, the method for imaging might incorporate one or several of the following features, taken in any technically admissible combination:
the ultrasound contrast agents present in the medium are ultrasound contrast agents functionalized with particles exhibiting electrical or optical properties.
the physical quantity is measured by using at least one electrode in electrical contact with the medium or an optical source and a detector.
the ultrasound contrast agents are microbubbles or vesicles containing a gaz.
at the imaging step, a contrast is associated to the image of the physical quantity in the medium, the power of each ultrasound wave applied at the step of applying being strictly inferior to the power of each ultrasound wave applied in a method for imaging the medium wherein ultrasound contrast agents are not present which provides an image of the physical quantity in the medium with the same contrast.
at the imaging step, super-resolution imaging techniques are used to locate electrical or optical quantities associated to single microbubble localization.
at the step of applying, each ultrasound wave is an unfocused wave or a non-single beam focused wave.
at the step of applying, each ultrasound wave propagates along a respective propagation direction, the propagation directions of each ultrasound wave being non-collinear.
the ultrasound contrast agents present in the medium have a diameter inferior to 5 microns.
a mean resonance frequency is defined for the ultrasound contrast agents present in the medium and each ultrasound wave has the same frequency, the frequency being a function of the mean resonance frequency of the ultrasound contrast agents.
at least one of the following properties is fulfilled :
   the medium is biological tissue;
   the medium is a muscle of an animal tissue; and
   the medium is a muscle, a brain or a myocardium.
the measuring step is carried out to obtain several measured physical quantities, notably several times, the imaging step being carried out for each measured physical quantity.
the imaging step comprises using a Radon inverse transformation.
the imaging step comprises using a technique based on retroprojection or spatiotemporal matched filtering or spatiotemporal inverse filtering.

The specification proposes a device for imaging a medium wherein ultrasound contrast agents are present, the device comprising an ultrasound transducer array adapted to apply at least one ultrasound wave to the medium, each ultrasound wave propagating up to the medium, the ultrasound transducer array being further adapted to collect each wave transmission of this at least one ultrasound wave. The device comprises a sensor adapted to measure physical quantity of an area, the physical quantity being affected by the or each applied ultrasound wave in the medium, the physical quantity being an electric quantity or an optical quantity. The device further comprises a controller adapted to image the physical quantity of the medium based on each wave transmission and the measured physical quantity.

The specification also concerns a device for imaging a medium wherein ultrasound contrast agents are present, the device comprising an ultrasound transducer array applying at least one ultrasound wave to the medium, each ultrasound wave propagating up to the medium, the ultrasound transducer array further collecting each wave transmission of this at least one ultrasound wave. The device also comprises a sensor measuring a physical quantity of an area, the physical quantity being affected by the or each applied ultrasound wave in the medium, the physical quantity being an electric quantity or an optical quantity. The device also comprises a controller adapted to image the physical quantity of the medium based on each wave transmission and the measured physical quantity.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 is a schematic view of an acoustoelectric imaging device;

FIG. 2 is a flowchart of an example of carrying out of a method for acoustoelectric imaging of an area; and

FIGS. 3 to 13 illustrate experimental results obtained by the Applicant.

### DETAILED DESCRIPTION OF SOME EMBODIMENTS

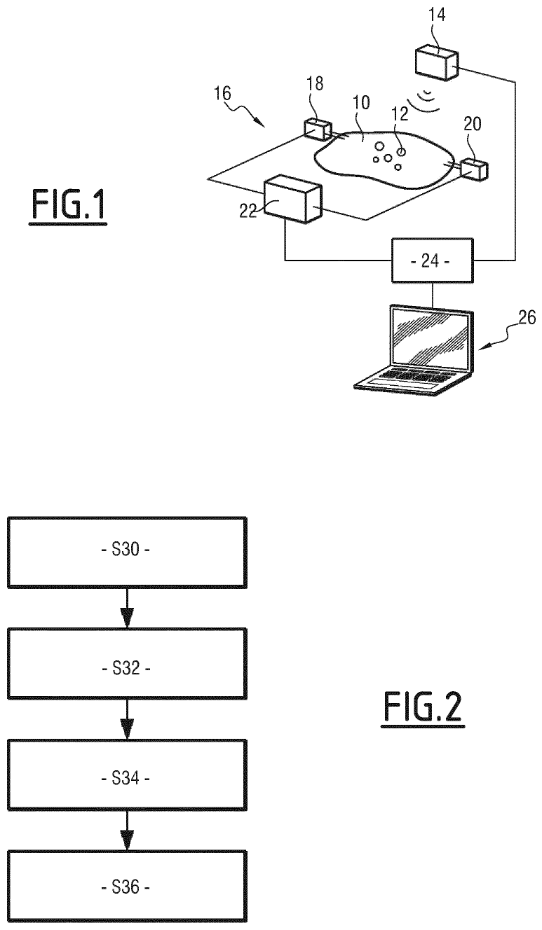

An example of acoustoelectric imaging device is represented on FIG. 1.

The acoustoelectric imaging device is adapted to image current density of a medium 10 wherein ultrasound contrast agents 12 are present.

The medium is a biological medium.

For instance, the medium is a medium pertaining to an animal, notably a mammal or a human being.

As a specific example, the medium 10 is biological tissues, in particular muscle, myocardium or brain.

The ultrasound contrast agents 12 are, for instance, bubbles.

In the remainder of the specification, as the bubbles have a small diameter, less than 1 centimeter, the bubbles are named microbubbles.

The microbubbles are, for example, the microbubbles described in an article by Dayton et al. whose title is “Molecular ultrasound imaging using microbubble contrast agent” in Frontiers in Bioscience 12, 5124-5142 dated September 2007.

For instance, each microbubble is a bubble of perfluorocarbon surrounded by lipid layers.

In variant, each microbubble is a vesicle containing a gaz.

In the remainder of the specification, the ultrasound contrast agents 12 are noted microbubbles 12.

The way by which the microbubbles 12 are present in the medium 10 is of no relevance for the invention, the imaging device being only used for imaging the medium 10, the preparation of the subject to be imaged pertaining to the domain of the scientists, notably the medical ones.

For information, the microbubbles 12 may be obtained by the scientists by injecting the microbubbles 12 in the medium 10 or by using a cavitation phenomenon induced by heating another location.

It should be noted that the cavitation is controlled so as to not damage surrounding tissues and respect the requirements of regulatory bodies such as the FDA. For example, the definity microbubbles must be used with a mechanical index below 0.8.

Such step is not achieved by the acoustoelectric imaging device.

According to the example of FIG. 1, the acoustoelectric imaging device comprises two kinds of sensors: an ultrasound transducer array 14 and an electric sensor 16.

The ultrasound transducer array 14 is a set of transducers adapted to emit incident ultrasonic waves and to collect ultrasonic waves.

For instance, the ultrasound transducer array 14 is a linear array formed by a set of transducers.

Alternatively, the ultrasound transducer array 14 is a two-dimensional array.

In this example, the ultrasound transducer array 14 comprises more than a hundred transducers, each transducer being adapted for obtaining a two-dimensional of the medium 10.

The electric sensor 16 is adapted to capture raw electrical signals during the propagation of the incident ultrasonic waves.

In the example of FIG. 1, the electric sensor 16 comprises a first pair of injecting electrodes 18 and a pair of measuring electrodes 20 linked to an amplifier 22.

The pair of injecting electrodes 18 is adapted to inject a current in the medium 10.

In variant, the current is endogenous.

The amplifier 22 is adapted to measure the difference in electric potential between the pair of measuring electrodes 20.

The evolution of the measured difference with time during the propagation of the incident ultrasonic waves is the raw electrical signals that the electric sensor 16 is adapted to capture.

The acoustoelectric imaging device further comprises a controller 24 and a display device 26.

The controller 24 is adapted to control the sensors 14 and 16.

The controller 24 is notably adapted to collect the signals acquired by the sensors 14 and 16. This implies that the controller 24 is adapted to control the ultrasound transducer array 14 and to obtain the signals collected by ultrasound transducer array 14. The controller 24 is also adapted to collect the raw electrical signals captured by the electric sensor 16.

In the described example, the controller 24 comprises analog-to-digital converters respectively connected to a transducer of the ultrasound transducer array 14 and the electric sensor 16, buffers respectively connected to the converters and a processor adapted to treat the signal stored in the buffers.

The display device 26 is adapted to display images, such as ultrasound images.

In the example of FIG. 1, the display device 26 is a computer provided with a keyboard and a screen.

The operating of the acoustoelectric imaging device is now described in reference to FIG. 2 which illustrates a flowchart of an example of carrying out of a method for imaging the medium 10.

It is assumed for the sake of exemplification that the density of microbubbles 12 in the medium 10 is superior to the density of microbubbles 12 that may be present naturally in the medium 10.

It is also assumed that the microbubbles 12 present in the medium 10 are microbubbles 12 with a diameter inferior to 5 microns.

This implies that a mean resonance frequency is defined for the microbubbles 12 which are present in the medium 10.

In the case of FIG. 2, the method for imaging comprises four steps: a step of applying S30, a step of measuring S32, a step of imaging S34 and a step of displaying S36.

At the step of applying S30, ultrasound waves are applied to the medium 10 by the ultrasound transducer array 14.

The number of applied ultrasound waves is superior or equal to 2.

Preferably, the number of applied ultrasound waves is inferior or equal to 100.

Each ultrasound wave propagates along a respective propagation direction.

In the specific proposed example, the propagation directions of each ultrasound waves are non-collinear.

At the step of applying S30, the mechanical index corresponding to the power of each ultrasound wave is inferior or equal to 1.5.

In any case, the power of each ultrasound wave applied at the step of applying being strictly inferior to the power of each ultrasound wave applied in a method for imaging the region of interest of the medium 10 wherein ultrasound contrast agents 12 are not present which provides an image with the same contrast.

This corresponds to the fact that the mechanical index in a conventional method (medium with no ultrasound contrast agent) is strictly superior to the mechanical index in the present method.

For instance, the mechanical index in a conventional method is equal to 1.9 while the mechanical index in the present method is equal to 1.1.

In such context, the contrast is associated to the map of the physical quantity in the medium 10.

Furthermore, each ultrasound wave is an unfocused wave.

An unfocused ultrasound wave is a wave for which an aperture is defined.

The aperture has a specific size labeled D.

An ultrasound wave is considered as unfocused if the minimal width Wₘᵢₙ of the ultrasound beam associated to the ultrasound wave at a depth F is larger than the ratio of the product of the wavelength λ of the ultrasound wave by the depth F with the specific size D of the aperture.

This means that the unfocused waves are plane waves or divergent waves.

For instance, each wave is a non-single beam focused wave.

In the specific example described, each ultrasound wave has the same frequency, the frequency being a function of the mean resonance frequency of the microbubbles 12.

For instance, the function is a linear function or more generally a polynomial function.

At the step of measuring S32, the current density in the medium 10 is measured by the electric sensor 16.

The step of measuring S32 is achieved at least during the propagation of the waves applied at the step of applying S30.

This means that at least one measure of the step of measuring S32 is carried out in the medium 10 wherein ultrasound waves propagate and wherein ultrasound contrast agents are present.

In this specific example, the measuring step is carried out several times to obtain several measured current densities.

At the step of imaging S34, the current density of the medium 10 is imaged based on each propagation direction and each measured current density.

The step of imaging S34 is to be construed as an image formation step enabling to reconstruct a map of the current density in the medium 10.

The term “image” is also used to designate such map.

For this, the step of imaging S34 enables to correlate the spatial information linked to the propagation directions with the quantitative information linked to the measured current densities.

Such correlation is made by using a Radon inverse transformation or a technique based on retroprojection.

According to another embodiment, such imaging step comprises achieving a superlocalization of the microbubbles 12 as described in document U.S. Pat. No. 9,329,260 and using this result to obtain the map.

At the end of the step of imaging S34, a map of the current density in the medium 10 is obtained.

At the step of displaying S36, the map of the current density in the medium 10 is displayed on the display device 26.

Such method for imaging enables to obtain improved resolution in the current density maps of the medium 10.

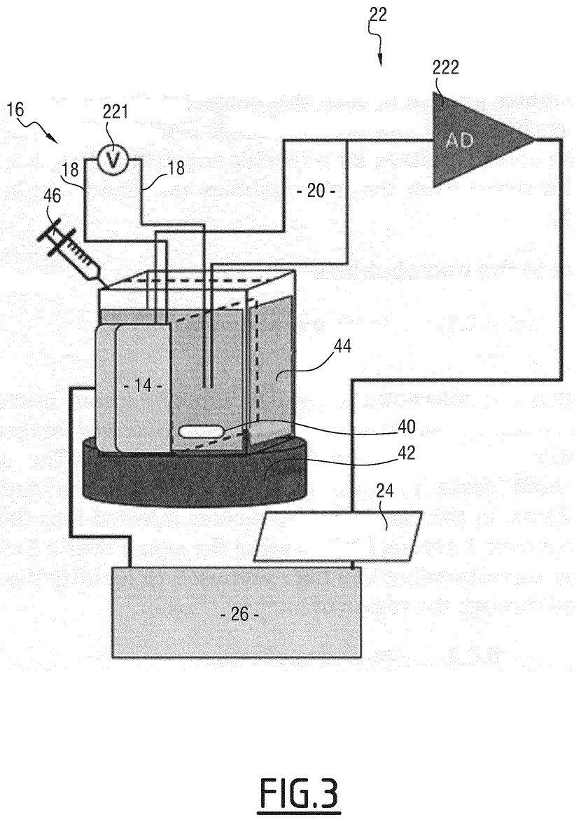

This improved resolution has been experimentally shown by the Applicant in reference to the results illustrated by FIGS. 3 to 13.

Measurements were performed using a saline phantom in which two current-injecting electrodes were placed between two measuring electrodes. Signal from the measuring electrodes was fed via high common-mode rejection ratio amplifier to a single channel of the Vantage ultrasound system. The acoustoelectric effect was triggered by an ultrasound wave emitted perpendicularly to the current distribution generated by the electrodes using a linear 5 MHz array connected to channels 1 to 128 of a Vantage system. Bubbles of diameters 2.5 μm on average were injected into the saline solution, and contrast was measured for microbubble concentrations of 1/1000000 to 5/100 and probe frequency comprised between 3 MHz and 6 MHz. Images were acquired for a saline phantom and in-vivo in an isolated rat heart placed in a Langendorff system.

The Applicant has notably shown that injection of microbubbles led to an increased in contrast at a peak pressure of 0.5 MPa in the saline phantom, and allowed for in-vivo measurement of the electrical activity in a rat heart. The concentration of microbubbles indicated an optimal value of 1/1000, with lower contrast for very low and very high concentrations. The effect was largest when emitting at 3.4 MHz, as a trade-off between the microbubbles resonating frequency and the probe bandwidth.

Further details relative to the experience carried out are to be found in the experimental section.

These results show that the presence of microbubbles improves the contrast of an acoustoelectric imaging method.

As a consequence, the proposed method for imaging is a contrast-enhanced acoustoelectric imaging method.

In addition, the mechanical index corresponding to the power of each applied ultrasound wave can be reduced to obtain a map with the same resolution. This is beneficial to the subject, notably when the method is carried in vivo.

In another embodiment, the microbubbles 12 are functionalized with particles exhibiting electrical properties.

This means for the specific examples described that the most outer lipid layer is functionalized with particles exhibiting electrical properties.

For functionalizing, Polyethylene glycol (PEG) may be deposit on the most outer lipid layer, the PEG being able to accept any particles exhibiting electrical properties.

Examples of particles exhibiting electrical properties in the context of microbubbles is, for instance, known from an article by S. Sersi and M. Borden whose title is “Microbubble Compositions, Properties and Biomedical Applications” Bubble Sci Eng Technol. 2009 November; 1(1-2): 3-17.

This enables to obtain an increased sensitivity of the method for imaging.

Notably, super-resolution imaging techniques can be to locate electrical property associated to single bubble localization with a spatial accuracy lower than the ultrasonic wavelength.

The term “super-resolution” stands for any method with a spatial accuracy lower than the spatial accuracy obtained by the Rayleigh criteria that is lower than the ultrasonic wavelength.

In another embodiment, at the step of measuring S36, a physical quantity distinct from the current density is measured.

The physical quantity is, for instance, another electrical quantity, notably one for which electrical current is endogenous or externally applied.

In variant, the physical quantity is an optical quantity. In such case, the method for imaging is an acousto-optic method. In such case, an optical source and a detector may be implied.

In each embodiment, the method for imaging is a method for imaging a medium 10 wherein microbubbles 12 are present, the method comprising at least the step of applying at least one ultrasound wave to the medium 10, each ultrasound wave propagating up to the medium 10, to collect each wave transmission of this at least one ultrasound wave. The method for imaging also comprises a step of measuring a physical quantity in the medium 10, the physical quantity being affected by the or each applied ultrasound wave in the medium 10, the physical quantity being an electric quantity or an optical quantity, and a step of imaging the physical quantity of the medium 10 based on each wave transmission and the measured physical quantity.

In other words, the method for imaging is an acousto-optic or an acoustoelectric imaging method applied to a medium 10 with microbubbles 12.

Such method for imaging enables to map a physical quantity of a medium, notably current densities, with an improved resolution.

The improvement in resolution is at this stage not fully understood.

Several hypotheses may be made.

One hypothesis is that the ultrasound contrast agents enable to obtain a better resolution in the location of the ultrasound contrast agents with ultrasound waves, this resulting in a better location of the acoustoelectric signal provided the acoustoelectric signal mainly comes from the ultrasound contrast agents.

Another hypothesis is that the ultrasound contrast agents enable to obtain a better acoustoelectric effect, this facilitating the detection of the associated signal.

Still another hypothesis is that both previous hypotheses are to be combined, this resulting in a complex interaction.

The embodiments and alternative embodiments considered here-above can be combined to generate further embodiments of the invention.

Experimental Section

The experimental section is devoted to show experiments made by the Applicant and to discuss the obtained results.

INTRODUCTION

Imaging the electrical activation of biological tissues in crucial in applications such as cardiology, to provide better understanding for the diagnosis and treatment of pathologies such as arrhythmias. Ultrafast Acoustoelectric Imaging (named UAI in what follows) has been recently proposed as a novel technique for non-invasive direct imaging of electrical activation, showing high accuracy and time resolution in-vitro. UAI is based on the acoustoelectric effect, in which the propagation of an acoustic pressure wave through a medium locally modifies its impedance. This phenomenon can be measured using electrodes to detect the impedance change when the ultrasound wave location coincides with the current density inside the medium. The measured signal occurs at the intersection of the pressure wave and the current density. Knowing the ultrasound wave position assuming a constant speed of sound, it is therefore possible to accurately localize and characterize the current density, producing high quality images in two dimensions and three dimensions. One of the main challenges in UAI is the ability to detect very small current densities of the same order of magnitude as the ones occurring in-vivo. Although previous work has shown the high sensitivity of the method, increased image contrast would allow for easier and more accurate imaging of the biological current densities, which is particularly important for generating real-time maps of the electrical activation.

Bubbles exist as a contrast agent for ultrasound imaging, in which the pressure wavefront triggers a volume oscillation of the microbubbles. Their behavior has been extensively studied in the literature. Since the microbubbles' reflective properties are directly linked to their size, this produces an increased signal at the localization of each microbubble, thereby improving the image contrast in regions where these are present. In UAI, the amplitude of the signal is also directly linked to the pressure peak generated by the ultrasound transducer. It is therefore hypothesized that the UAI contrast can be increased by the addition of microbubbles into the imaged medium. The present work aims at demonstrating the impact of adding microbubbles to the medium images with UAI, and identifying optimal parameters for use of microbubbles as contrast agents for imaging current densities.

Methods

Experimental Setup

A Vantage ultrasound system (Verasonics Inc., USA) including two 128-channels connectors was used to a) generate the ultrasound wave, b) process the UAI signal and c) if necessary record the ultrasound echo from the probe and produce a B-mode image.

FIG. 3 describes the experimental setup used for contrast-enhanced UAI. The current was generated via a pair of copper-wire electrodes 18 placed in a pool of conductive saline solution 44 which can be stirred by a magnetic stirrer 40 controlled by a stirring plate 42. The current generated is measured by a voltmeter 221. The 5.2-MHz central frequency ultrasound transducer was connected to one of the connectors from the Vantage system, and placed in front of a Mylar window in the wall of the saline pool. The ultrasound plane wave was generated between the current electrodes in a plane perpendicular to the current density. A pair of measuring electrodes 20 was placed on both sides and on the same plane with the current generating electrodes 18. These measured the voltage difference induced by the ultrasound wave propagating through the current density, using a high-common mode rejection ratio differential amplifier 222. This signal was then fed back to the computer via a single channel from a break-out board connected to the second connector of the Vantage system. The system also allowed for the recording of standard Ultrafast Ultrasound Imaging (UUI) data to produce two-dimension echography images of the imaged object's structure. The microbubbles were injected into the saline solution with a syringe, and a magnetic stirrer at the bottom of the pool (controlled by a stirring plate underneath) allowed for constant mixing of the solution.

In FIG. 3, the electrodes plane and the ultrasound wave emission plane are indicated in dashed lines.

Characterization of the Microbubbles

Description of the Microbubble Solution

Bubbles were obtained from SonoVue® as a 8 μL/ml solution of lipid microspheres containing sulfur hexafluoride with a mean diameter of 2.5 μm. The resonance frequency of the microbubbles is approximately 3 MHz. The microbubble solution was drawn with a syringe from the commercial vials, and injected into a saline volume during the experiments according to the concentration targeted. An additional needle was inserted into the vial before drawing the microbubbles and removed after, to allow air flow between the vial's contents and the air in the room. Because of the small volumes needed, the microbubbles solution drawn from the vial was first deposited onto a petri dish, allowing for the desired amount to be drawn with a precision pipette, and finally injected into the saline pool where the solution was mixed with the magnetic stirrer 40.

Optimal Probe Voltage

This experiment was run to determine the highest peak pressure to be emitted in order to ensure stable microbubble concentration in the solution. Indeed, microbubbles tend to collapse above a certain level of pressure applied to them. The microbubbles were imaged using UUI with 17 angles at pressure peaks ranging from 0.22 MPa to 1.7 MPa for a duration of 4.5 s and a frame rate of 500 Hz, at a concentration of 1/10000. The number of microbubbles present in each UUI image was measured using a script for locating each microbubble within a single pixel in the image, via Singular Value Decomposition. The optimal voltage for a specific acquisition length L was defined as the value for which the signal from the microbubbles remained within 10% of its initial value for the time L.

Effect of the Microbubbles

In-Vitro Experiments

The UAI signal was measured in the saline pool without microbubbles and with microbubbles at a concentration of 1/10000. The solution was imaged for a probe peak pressure of 0.5 MPa during 1 second with 9 plane waves. The data obtained was reconstructed in both cases to form a two dimensional image and averaged over 10 frames corresponding to 25 ms. In the case of microbubbles injected into the medium, the UAI signal was measured over one second by averaging the signal over a 5×5 region of interest for each frame. The corresponding UUI data was used to identify the frames in which a microbubble passed through the region of interest chosen.

In-Vivo Application

An isolated Langendorff heart system was used to image a 450-g male adult rat heart using UAI. Bubbles were injected at a concentration of 1/10000 within the flow of nutritive Krebs-Henseleit solution feeding the heart within the Langendorff system. UAI images were acquired for 18 s at a probe peak pressure of 1 MPa.

Contrast Enhancement Measurements

Impact of Frequency

The impact of the probe frequency on the image contrast with microbubbles was investigated by varying the probe frequency between 3 MHz and 6 MHz while performing UAI measurements with a fixed microbubble concentration of 1/10000. The current injected was 27 V and images were acquired at a frame rate of 500 Hz for 0.6 s with a peak pressure of 0.7 MPa. The contrast was measured as the peak value of the acoustoelectric signal (average of a 5-pixel region centered on the maximum) divided by the average intensity in a 15-by-15 region close to the signal spot chosen in the background. The background region was chosen just above or just under the signal so as to avoid measuring any potential side lobes. Measurements were made on single frames and subsequently averaged over 100 frames.

Impact of Microbubble Concentration

The microbubble concentration in the saline solution was varied between 5/1000 and 1/1000000 in order to evaluate the impact of the concentration on the UAI contrast. Volumes between 0.2 μL and 1 mL were injected into the 200 mL saline pool. Measurements were performed for an intermediate number of 9 plane waves to ensure good resolution while maintaining a small volume of data. The current injected was 27 V and the ultrasound wave was emitted with a peak pressure of 0.7 MPa, at its central frequency of 5.2 MHz. Contrast was measured as described above.

Results

Characterization of the Microbubbles

Optimal Pressure Peak

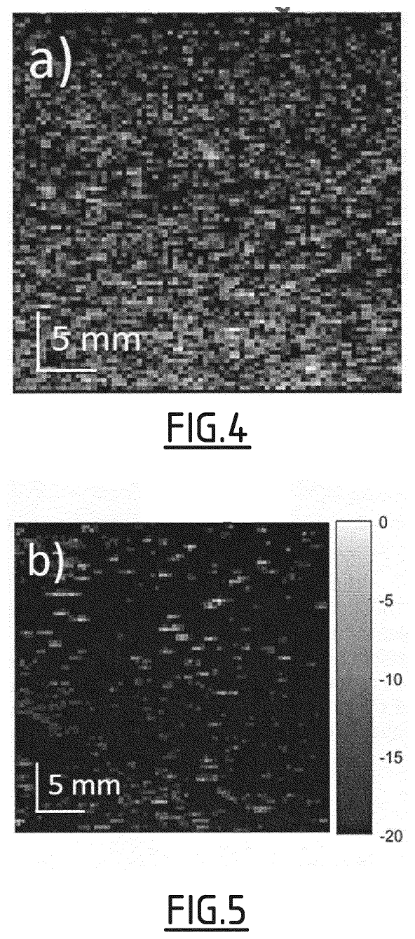

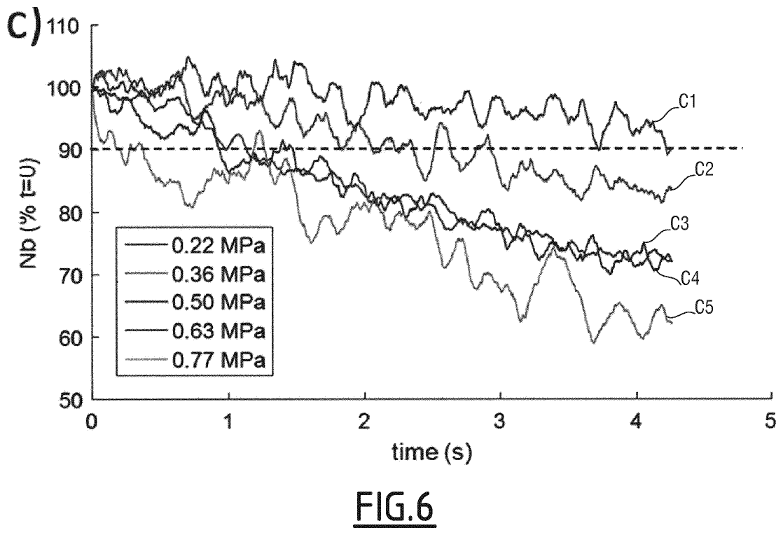

FIGS. 4 to 6 show a measure of the number of microbubbles present in the field of view for B-mode images acquired at pressure peak values varying between 0.22 MPa (see FIGS. 4) and 0.77 MPa (see FIG. 5) for 4000 frames corresponding to 4.3 s. The quantity of microbubbles decreased with time, to lower levels for higher pressure peaks (see FIG. 6 and the five curves C1, C2, C3, C4 and C5). For a pressure peak applied of 0.22 MPa, the number of microbubbles was still within 10% of the initial value after 4 seconds, whereas this number decreased by about 20% within 1 s for a peak pressure of 0.77 MPa. The maximum peak pressure for which the number of microbubbles remained within 10% was 0.63 MPa and 0.36 MPa respectively for acquisitions of 2 s and 1 s respectively.

Effect of the Microbubbles

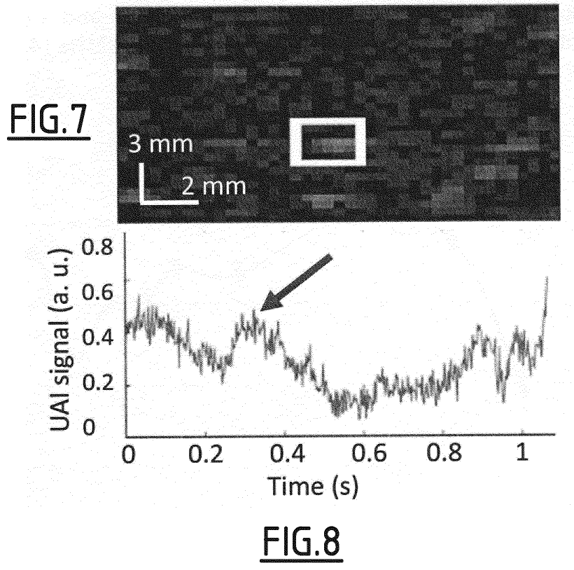

In FIGS. 7 and 8 are represented UUI of microbubbles in medium (FIG. 7) and UAI signal over time (FIG. 8), measured within a region of interest indicated by a white window on the top panel. The arrow points to the time frame corresponding to the top image.

This shows the UAI signal over time measured within a specific region of interest. The associated UUI image shows that the UAI signal increases by a factor of two approximately when a microbubble is located within this region of interest.

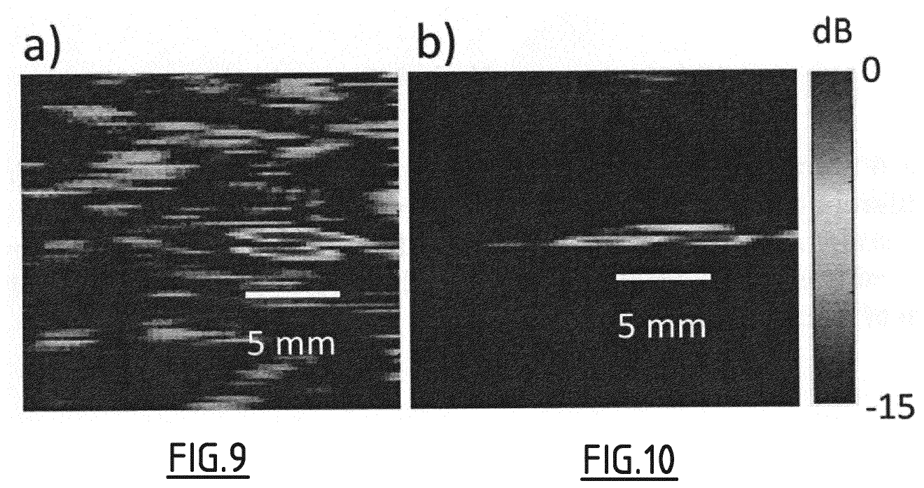

FIG. 9 corresponds to the UAI signal averaged over 10 frames obtained without microbubbles while FIG. 10 corresponds to the UAI signal averaged over 10 frames obtained with microbubbles at a concentration of 1/10000.

The comparison of the UAI signal without and with microbubbles in both FIGS. 9 and 10, when averaged over 10 frames and displayed in dB shows a higher amount of noise is visible on the panel representing the signal without microbubbles, which indicates a lower contrast value.

Contrast Enhancement Measurements

Impact of Frequency

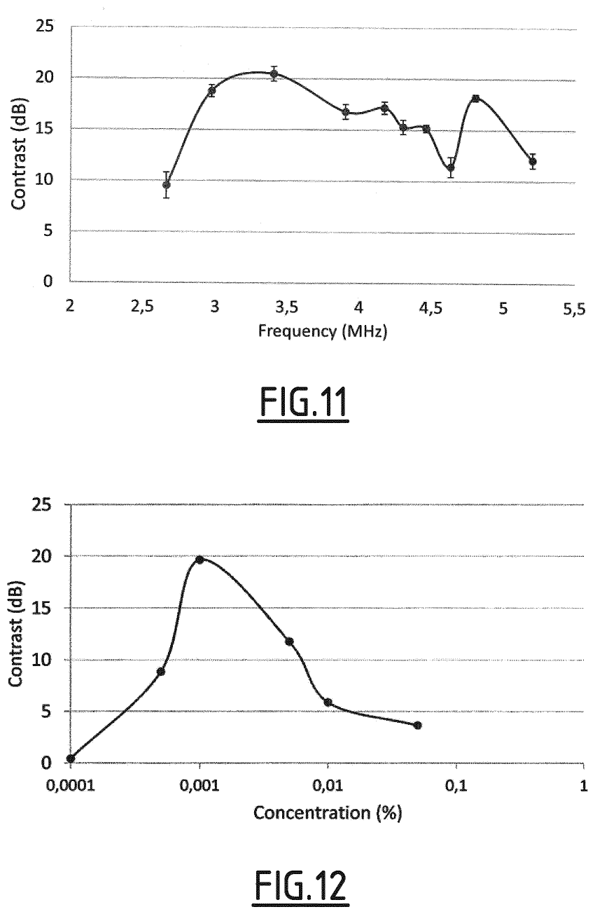

FIG. 11 shows the contrast measured on UAI images acquired at a fixed microbubble concentration ( 1/10000) for varying probe frequency values. There is an increase in the contrast leading to two visible contrast peaks at 3.4 MHz and 4.8 MHz. It should be noted that the microbubbles' resonating frequency is around 3 MHz.

Impact of Microbubble Concentration

FIG. 12 shows the contrast measured on UAI images acquired with microbubble injected at different concentrations in the medium, with the concentration displayed on a logarithmic scale. A peak was obtained around 1/1000, while 20 times lower and 5 times lower contrast was obtained for very low (0.001%) and very high (5%) concentration of microbubbles respectively.

In-Vivo Application

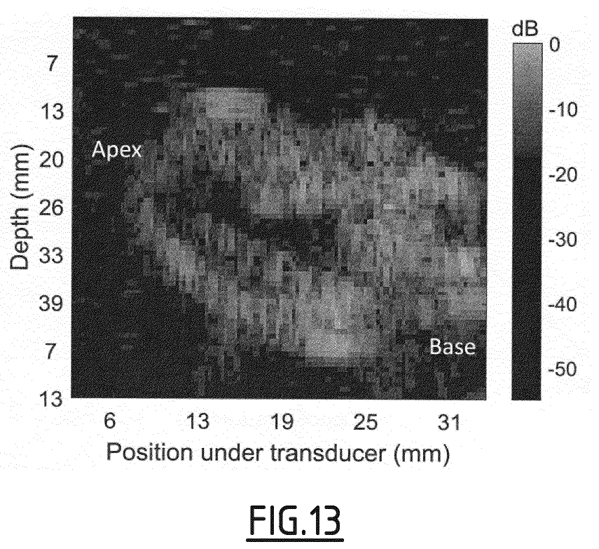

FIG. 13 shows the UUI image of the isolated heart on a single frame, with the overlayed UAI signal, averaged over 6 s, obtained after the injection of microbubbles. The scale is 0 dB to −55 dB, and 0 dB to −10 dB UI and UAI respectively.

Discussion and Conclusion

Bubbles were successfully injected into the solution and imaged using the UUI images. The number of microbubbles in the field of view of the probe decreased over time, at a rate corresponding to the level of the pressure peak applied, but remained stable over several seconds for a pressure peak of 0.6 MPa. At higher pressures, the microbubbles experience a collapse phenomenon, which has been described in several published studies. However reducing the pressure peak also reduces the magnitude of the acoustoelectric effect, which is directly linked to the ultrasound pressure for a given current density.

The effect of the microbubbles was observed using the high temporal resolution of the UUI and UAI techniques, as an increase in the UAI signal at a specific location in the image when it is traversed by a microbubble. In addition, for a given probe peak pressure, averaged reconstructed UAI data shows higher contrast when microbubbles have been added to the imaged medium.

In-vivo use of the microbubbles for imaging the electrical activation in an isolated rat heart showed high contrast (<10 dB) when injecting microbubbles and imaging at a pressure peak of 1 MPa. This value is low compared to values of 3.5 MPa typically needed to obtain UAI signal without microbubbles in-vivo.

The UAI showed increased signal for probe frequencies between 3 MHz and 3.8 MHz and around 5 MHz. The latter is close to the central frequency of the probe used, for which the pressure emitted by the transducer is maximal (for a given voltage). It is therefore expected, since the acoustoelectric effect is directly linked to the pressure wave emitted, that the signal be higher at that frequency. However, even higher contrast values were obtained for lower frequencies, which are just above the microbubbles resonance frequency, around 3 MHz. In this case, the signal is increased not because of an increase acoustoelectric effect, but because of an increased contrast enhancement from the microbubbles. Although both these effects increase the UAI signal, the contrast seems enhanced mostly and for a larger range of frequencies when the microbubbles are close to their resonance frequency, suggesting that contrast loss from emitting at a frequency non-optimal for the probe is more than compensated by the microbubbles' resonance.

The UAI signal contrast was found higher for an intermediate microbubbles concentration value of 1/1000, while it decreased for lower and higher concentration values. The reduction of the signal (down to almost no contrast) for low concentrations was expected, since the contrast enhancement is then limited by the small number of contrast enhancing microbubbles. Conversely, for very high concentrations, the proportion of microbubbles in the solution makes it difficult for the both the ultrasound wave and the current to propagate, thereby limiting the acoustoelectric effect itself.

These results show the potential of microbubbles for enhancing UAI contrast, leading to high quality images even at low peak pressures.

1. A method for imaging a medium wherein ultrasound contrast agents are present, the method comprising at least the step of:
applying at least one ultrasound wave to the medium, each ultrasound wave propagating up to the medium, to collect each wave transmission of this at least one ultrasound wave,
measuring a physical quantity of the medium, the physical quantity being affected by the or each applied ultrasound wave in the medium, the physical quantity being an electric quantity or an optical quantity, and
imaging the physical quantity in the medium based on each wave transmission and the measured physical quantity.

2. The method according to claim 1, wherein the ultrasound contrast agents present in the medium are ultrasound contrast agents functionalized with particles exhibiting electrical or optical properties.

3. The method according to claim 2, wherein at the imaging step, super-resolution imaging techniques are used to locate electrical or optical quantities associated to single microbubble localization.

4. The method according to claim 1, wherein the ultrasound contrast agents are microbubbles or vesicles containing a gaz.

5. The method according to claim 1, wherein at the imaging step, a contrast is associated to the image of the physical quantity in the medium, the power of each ultrasound wave applied at the step of applying being strictly inferior to the power of each ultrasound wave applied in a method for imaging the medium wherein ultrasound contrast agents are not present which provides an image of the physical quantity in the medium with the same contrast.

6. The method according to claim 1, wherein the physical quantity is measured is measured by using at least one electrode in electrical contact with the medium (-1-0) or an optical source and a detector.

7. The method according to claim 1, wherein at the step of applying, each ultrasound wave is an unfocused wave.

8. The method according to claim 1, wherein at the step of applying, each ultrasound wave propagates along a respective propagation direction, the propagation directions of each ultrasound wave being non-collinear.

9. The method according to claim 1, wherein the ultrasound contrast agents present in the medium have a diameter inferior to 5 microns.

10. The method according to claim 1, wherein a mean resonance frequency is defined for the ultrasound contrast agents present in the medium and each ultrasound wave has the same frequency, the frequency being a function of the mean resonance frequency of the ultrasound contrast agents.

11. The method according to claim 1, wherein at least one of the following properties is fulfilled:
the medium is biological tissue;
the medium is a muscle of an animal tissue; and
the medium is a muscle, a brain or a myocardium.

12. The method according to claim 1, wherein the measuring step is carried out to obtain several measured physical quantities, notably several times, the imaging step being carried out for each measured physical quantity.

13. The method according to claim 1, wherein the imaging step comprises using a Radon inverse transformation.

14. The method according to claim 1, wherein the imaging step comprises using a technique based on retroprojection or spatiotemporal matched filtering or spatiotemporal inverse filtering.

15. A device for imaging a medium wherein ultrasound contrast agents are present, the device comprising:
an ultrasound transducer array applying at least one ultrasound wave to the medium, each ultrasound wave propagating up to the medium, the ultrasound transducer array further collecting each wave transmission of this at least one ultrasound wave,
a sensor measuring a physical quantity of an area, the physical quantity being affected by the or each applied ultrasound wave in the medium, the physical quantity being an electric quantity or an optical quantity, and
a controller imaging the physical quantity of the medium based on each wave transmission and the measured physical quantity.

16. A method for imaging a medium, the method comprising:
injecting ultrasound contrast agents in the medium,
applying at least one ultrasound wave to the medium, each ultrasound wave propagating up to the medium, to collect each wave transmission of this at least one ultrasound wave,
measuring a physical quantity of the medium, the physical quantity being affected by the or each applied ultrasound wave in the medium, the physical quantity being an electric quantity or an optical quantity, and
imaging the physical quantity in the medium based on each wave transmission and the measured physical quantity.
